(19) United States
(12) Patent Application Publication
(10) Pub. No.: US 2006/0025643 A1
(43) Pub. Date: Feb. 2, 2006

(54) Method for the production of synthetic hydrocarbons

(75) Inventors: Hans Peter Rath, Grunstadt (DE); Arno Lange, Bad Durkheim (DE); Helmut Mach, Heidelberg (DE)

Correspondence Address: OBLON, SPIVAK, MCCLELLAND, MAIER & NEUSTADT, P.C., 1940 DUKE STREET, ALEXANDRIA, VA, 22314, US

(73) Assignee: Basf Aktiengesellschaft, Ludwigshafen (DE)

(21) Appl. No.: 10/524,360

(22) PCT Filed: Aug. 11, 2003
(86) PCT No.: PCT/EP03/08903

§ 371 Date: Jun. 30, 2005

(30) Foreign Application Priority Data
Aug. 12, 2002 (DE) 10236927.5

(51) Int. Cl. C07C 2/02

(52) U.S. Cl. 585/522

(57) ABSTRACT

The present invention relates to a process for preparing synthetic hydrocarbons which comprises the oligomerization of 1-olefins having from 8 to 30 carbon atoms.

The present invention relates to a process for preparing synthetic hydrocarbons which comprises the oligomerization of 1-olefins having from 8 to 30 carbon atoms.

Hydrocarbons based on 1-olefins having from 8 to 30 carbon atoms are used as base oils in partly synthetic or fully synthetic motor oils. The preparation of these base oils comprises the oligomerization of 1-olefins having from 8 to 30 carbon atoms as central step. In most cases, the oligomerization is carried out in the presence of boron trifluoride and a protic compound which can form a complex with boron trifluoride and is usually referred to as activator, promoter or cocatalyst. Activators which have been mentioned in the prior art are, in particular, alcohols (cf. U.S. Pat. No. 3,149,178, U.S. Pat. No. 3,382,291, U.S. Pat. No. 3,780,128, U.S. Pat. No. 4,045,507, U.S. Pat. No. 4,045,508, U.S. Pat. No. 5,284,988, U.S. Pat. No. 5,498,815, EP-A 349 276 and EPA 1 006 097), water (U.S. Pat. No. 3,763,244, EP-A 349 276, EP-A 1 006 097) and carboxylic acids (U.S. Pat. No. 3,149,178). As alternatives, aprotic compounds such as ethers, esters of aliphatic carboxylic acids, ketones, aldehydes and acid anhydrides have also been proposed as activators, promoters or cocatalysts.

However, the oligomerization of 1-olefins in the presence of boron trifluoride and a protic activator as described in the prior art has a series of disadvantages. Firstly, it is necessary to work at high concentrations of free BF₃ in order to achieve a sufficient catalyst activity and a satisfactory conversion of the 1-olefins. In general, the reaction mixture therefore has to be supersaturated with boron trifluoride in the oligomerization and the reaction has to be carried out at high boron trifluoride pressures in the reactor, in general ≧2 bar. The consumption of boron trifluoride is consequently high and cannot be reduced below 2% by weight, based on the amount of 1-olefin used, even in the case of complicated recirculation measures. The high consumption of boron trifluoride is disadvantageous from two points of view, since costs are incurred both for the boron trifluoride itself and for the disposal of the fluorine-containing wastewater obtained in the work-up.

A further disadvantage is that base oils having a viscosity of 4 mm²/sec which is of particular economic interest (also referred to as PAO4) are in this way obtained only as coproducts together with base oils of higher viscosities, e.g. from 6 to 8 mm²/sec. Viscosity figures indicated here and in the following refer to the kinematic viscosity in mm²/sec of the base oils at 100° C. determined using an Ubbelohde viscometer in accordance with DIN 51562-1 to 4, unless indicated otherwise. Base oils having a viscosity of 5 mm²/sec (PAO5) have hitherto not been obtainable in this way.

The isolation of PAO4 is carried out by fractional distillation in which unreacted 1-olefin and dimer are separated off first and PAO4, which comprises predominantly trimers and tetramers, is subsequently distilled off from products having a higher viscosity. This results in an additional increase in the production costs for PAO4. In addition, the PAO4 obtained as distillate has only a moderate viscosity index, which is particularly undesirable for use of PAO4 in light duty motor oils.

There have been many attempts to solve the problems indicated here. Thus, for example, U.S. Pat. No. 5,284,988 and U.S. Pat. No. 5,498,815 describe processes for preparing synthetic hydrocarbons in which a 1-olefin is firstly dimerized. This dimer is subsequently isomerized and then reacted with a further 1-olefin in the presence of a boron trifluoride catalyst. In this way, a PAO4 having a high viscosity index can be produced. However, this process is very complicated.

WO 01/21675 describes a process in which a 1-olefin is firstly oligomerized in the presence of transition metal catalysts, the low molecular weight fraction is separated off from this and the low molecular weight fraction is subsequently reacted in the presence of an acid catalyst, for example a boron trifluoride-alcohol complex. Firstly, the metallocene catalysts used are expensive and, secondly, this also gives PAO4 together with coproducts of higher viscosity.

It is an object of the present invention to provide a process for preparing synthetic hydrocarbons by oligomerization of 1-olefins in the presence of boron trifluoride catalysts which makes do with a reduced boron trifluoride consumption. In addition, the process should be suitable for preparing hydrocarbons having viscosities of from 4 mm²/sec (=PAO4) to 5 mm²/sec (PAO5) without the desired product having to be distilled.

We have found that this object is achieved by a process in which the oligomerization is performed at an absolute pressure of boron trifluoride in the reactor of not more than 1.3 bar, and in which the reaction mixture used for the oligomerization comprises boron trifluoride and at least one protic activator compound A1 (hereinafter referred to as activator) and also at least one further compound which is selected from among
aprotic organic compounds A2 which are selected from among ethers, comprising at least one ether oxygen atom, and
halogenated hydrocarbons, in particular chlorinated hydrocarbons having from 1 to 6 carbon atoms and 1, 2 or 3 halogen atoms, in particular chlorine atoms,
   where the molar ratio of boron trifluoride to the total amount of compounds A1 and A2 is in the range from 0.8:1 to 4:1.

The present invention accordingly provides a process for preparing a synthetic hydrocarbon, which comprises the oligomerization of 1-olefins having from 8 to 30 carbon atoms in the presence of boron trifluoride and at least one protic activator A1, wherein the reaction is carried out in the presence of at least one further compound selected from among the above-defined aprotic organic compounds A2 and the above-defined halogenated hydrocarbons, in particular chlorinated hydrocarbons and the molar ratio of boron trifluoride to the total amount of compounds A1 and A2 is in the range from 0.8:1 to 4:1 and wherein the absolute pressure of boron trifluoride in the reactor does not exceed a value of 1.3 bar.

It is assumed that the presence of the compound A2 or the halogenated hydrocarbon in the reaction mixture increases the activity of the catalytically active boron trifluoride species, possibly as a result of improved solubility of the catalytically active species in the reaction mixture and/or as a result of an increase in the acidity of the catalytically active species, so that a better conversion is achieved at the same BF₃ concentration. Presumably for these reasons, a high boron trifluoride concentration in the reaction mixture (and consequently a high boron trifluoride pressure in the reactor) is not necessary to achieve satisfactory conversions. In addition, high conversions of the 1-olefins used can also be achieved in this way without increased formation of higher oligomers occurring, so that low-viscosity products can be produced in a targeted manner in this way.

The process of the present invention is advantageously carried out at BF₃ concentrations which do not exceed, or only insignificantly exceed, the saturation concentration of BF₃ in the reaction mixture at the reaction temperature at normal pressure, i.e. a BF₃ partial pressure of 1013 mbar, at least at the beginning of the reaction. The concentration of BF₃ in the reaction mixture is preferably not more than 120% of this saturation concentration and is in particular at the saturation concentration and particularly preferably below, i.e. not more than 90% of, the saturation concentration. The saturation concentration can be determined by a person skilled in the art in a simple manner by passing BF₃ into a particular volume of the reaction mixture until saturation is achieved at atmospheric pressure and weighing the BF₃ introduced.

Accordingly, the absolute pressure of boron trifluoride in the reactor (i.e. the partial pressure of boron trifluoride in the gas phase in the reactor) is not more than 1.3 bar, preferably not more than 1.2 bar, in particular not more than 1.1 bar, more preferably not more than 1.05 bar, especially not more than 1 bar, e.g. 0.5 to 1.1 bar, especially 0.8 to 1.05 bar. The pressure of boron trifluoride in the reactor can be adjusted by conventional means, for example by charging the reactor with boron trifluoride at a predetermined partial pressure, e.g. by charging a mixture of BF₃ and an inertgas into the reactor and/or by adjusting the charging rate in a way, that saturation concentration is not achieved.

The boron trifluoride concentration in the reaction mixture is therefore generally less than 3% by weight, preferably less than 2% by weight and is particularly preferably in the range from 0.1 to <2% by weight, in particular in the range from 0.3 to 1.8% by weight and especially in the range from 0.5 to 1.5% by weight, based on the total weight of the reaction mixture. If phase separation into a first phase having a high boron trifluoride concentration and a second phase having a low boron trifluoride concentration occurs during the reaction, these figures relate to the second phase. The total concentration of BF₃ in the reaction mixture can then also be higher. However, a value of 10% by weight, in particular 5% by weight, is preferably not exceeded.

The molar ratio of boron trifluoride to the total amount of compound A1 and, if present, A2 is preferably not more than 2.5:1, in particular not more than 2:1 and particularly preferably not more than 1.8:1. This ratio is preferably at least 1:1 and in particular at least 1.2;1.

As compounds A1, it is in principle possible to use all protic activators known from the prior art which form a complex with boron trifluoride. These include, for example, water, aliphatic or aromatic alcohols, e.g. alkanols having from 1 to 20 carbon atoms, aliphatic polyols such as glycerol, glycol and pentaerythritol, hydrogen fluoride and carboxylic acids, e.g. aliphatic carboxylic acids such as acetic acid or aromatic carboxylic acids such as benzoic acid. Preferred activators A1 are selected from among alkanols having from 1 to 10 carbon atoms, in particular from 1 to 6 carbon atoms and especially from 2 to 4 carbon atoms, and among these preferably primary and secondary alkanols.

In the process of the present invention, the oligomerization is carried out in the presence of a compound A2 and/or in the presence of a halogenated hydrocarbon.

As compounds A2, it is in principle possible to use all aprotic, in particular aliphatic ethers, which have at least one, e.g. 1, 2, 3, 4 or 5 and preferably exactly one ether oxygen atom. Aliphatic means that the ether does not comprise aromatic or cycloaliphatic moieties. Preferably, the ether does not comprise further functional groups. Compounds A2 preferably have at least 5 carbon atoms, e.g. from 5 to 30 and in particular from 10 to 20 carbon atoms. Preferred compounds A2 are dialkyl ethers having at least 5 carbon atoms, e.g. from 5 to 30 and in particular from 10 to 20 carbon atoms. Examples of such compounds are methyl tert-butyl ether, ethyl tert-butyl ether, isopropyl tert-butyl ether, di-n-butyl ether, di-n-pentyl ether, diisopropyl ether, di-n-hexyl ether, di-n-heptyl ether, di-n-octyl ether and di-2-ethylhexyl ether. Likewise suitable are ether compounds of the general formula
R—[O-Alk]ₙ-O—R′

wherein R and R′ independently of each other are alkyl having from 1 to 4 carbon atoms, n is 1, 2, 3 or 4, in particular 1 or 2 and Alk is a linear or branched alkylene moiety having 2, 3 or 4 carbon atoms. Alkyl having from 1 to 4 C-atoms includes e.g. methyl, ethyl, n-propyl, n-butyl, 2-propyl etc. Alkylene having from 2 to 4 C-atoms include e.g. 1,2-ethanediyl, 1,2-propanediyl, 1,3-propanediyl, 1,2-butanediyl and 1,4-butanediyl.

Among the compounds A2, preference is given to those whose Gutmann donor number (V. Gutmann and E. Wychera, Inorg. Nucl. Chem. Lett., 1966, 2, 257) is in the range from 5 to 25, in particular in the range from 10 to 20. A review giving numerous values of donor numbers and describing methods of determining them may be found in Y. Marcus, Chemical Society Reviews 1993, pp. 409-416.

As halogenated hydrocarbons, in principle all aliphatic hydrocarbons having from 1 to 6 carbon atoms can be used, wherein 1, 2 or 3 hydrogen atoms are substituted by halogen atoms. Herein the term halogen includes in particular fluorine, chlorine and bromine, especially chlorine. Chlorinated hydrocarbons which may be mentioned are, for example, dichloromethane, trichloromethane, dichloroethane, trichloroethane, chloropropane, chlorobutane, chloropentane and the like. A preferred chlorinated hydrocarbon is dichloromethane. Chlorofluorocarbons and chlorobromocarbons are also suitable.

If the oligomerization is carried out in the presence of a chlorinated hydrocarbon, the concentration of the chlorinated hydrocarbon in the reaction mixture is preferably from 2 to 20% by weight and in particular from 5 to 15% by weight. Dichloromethane is in this case a particularly preferred chlorinated hydrocarbon.

If the oligomerization is carried out in the presence of a compound A2, the compounds A1 and A2 are generally used in a molar ratio of A1:A2 in the range from 5:1 to 1:20, preferably from 3:1 to 1:10 and in particular from 2:1 to 1:5.

The type and amount of the compound A2 are preferably chosen so that the mean donor number of compounds A1 and A2, weighted according to the mole fractions of A1 and A2, is in the range from 15 to 34, in particular in the range from 15 to 28.

If the oligomerization is carried out in the presence of a compound A2, it has been found to be advantageous for the mean number of carbon atoms in the compounds A1 and A2, weighted according to the mole fractions of A1 and A2, to be in the range from 4 to 8, in particular in the range from 4.5 to 7. The compound A1 is then preferably selected from among alkanols having from 2 to 6 and in particular from 2 to 4 carbon atoms, e.g. ethanol, n-propanol, isopropanol, n-butanol and 2-butanol. Under these conditions, the mixture to be oligomerized, boron trifluoride and the compounds A1, A2 and, if present, the halogenated hydrocarbon generally form a homogeneous phase. In this embodiment, the reaction can be carried out in the presence or absence of a chlorinated hydrocarbon.

In another embodiment of the present invention, the type and amount of compound A1 and the type and amount of any compound A2 used and the type and amount of any chlorinated hydrocarbon used are chosen so that the solubility of free BF₃ and the boron trifluoride bound by compounds A1 and A2 in the output from the reactor after the oligomerization is complete is less than 1% by weight, in particular less than 0.8% by weight. The solubility of the boron trifluoride in the output from the reactor is preferably at least 0.4% by weight and in particular at least 0.5% by weight. Under these conditions, a first phase having a high BF₃ concentration and a second phase which is low in BF₃ but rich in product or starting material are formed. The phases are present in the form of a fine emulsion which is largely stable for a period of 3 minutes.

The compound A1 is then preferably selected from among water, alkanols having from 1 to 3 carbon atoms and hydrogen fluoride. If a compound A2 is used, this is preferably selected from among dialkyl ethers having from 5 to 10, preferably from 5 to 8, carbon atoms.

Such a low solubility can be achieved, for example, by using ethanol, isopropanol or n-propanol as compound A1 in combination with a chlorinated hydrocarbon and in the absence of a compounds A2. As regards the type and amount of the chlorinated hydrocarbon, what has been said above applies.

As an alternative, such a solubility can also be achieved by using methanol, water or hydrogen fluoride as compound A1 and a dialkyl ether having at least 5, preferably from 5 to 8, carbon atoms as compound A2. The molar ratio of compound A1 to compound A2 is then preferably in the range from 1:2 to 1:20.

The boron trifluoride concentration in the reaction mixture in this embodiment is preferably from 0.5 to 10% by weight, in particular from 0.7 to 5% by weight. Boron trifluoride concentrations above 3% by weight do not adversely affect the success of the process of the present invention in this case, since, due to the phase separation into a first catalyst phase rich in boron trifluoride and a second starting material/product phase which is low in boron trifluoride, the catalyst can be separated off from the reaction mixture in a very simple manner and be returned to the oligomerization reaction. The activity of the boron trifluoride complex in the reaction mixture is nevertheless sufficiently high to effect rapid conversion, presumably because of the fine dispersion of the BF₃-rich phase in the low-BF₃ phase.

Without being tied to a theory, it is assumed that under these reaction conditions the oligomerization of the 1-olefin or the further reaction of the dimer takes place in the first phase having a higher BF₃ concentration, with 1-olefin or dimer continually diffusing from the second phase into the first phase and, conversely, the oligomerization product migrating from the first phase into the second phase because of its low solubility in the first phase. This results in an acceleration of the reaction in the complex-rich phase, which leads overall to higher conversions.

Starting materials used in the process of the present invention are 1-olefins, i.e. aliphatic, acyclic hydrocarbons having a terminal double bond which can have a vinyl structure (CH₂═CH—R) or a vinylidene structure (CH₂═CRR′), where R and R′ are saturated aliphatic, acyclic hydrocarbon radicals. According to the invention, the 1-olefins to be oligomerized preferably comprise at least 50% by weight, in particular at least 80% by weight and particularly preferably at least 90% by weight, of 1-olefins having a vinyl structure.

In particular, the starting materials used in the process of the present invention are 1-olefins having from 8 to 14 carbon atoms, in particular with a view to preparing oligomers having a viscosity of from about 4 mm²/sec to about 5 mm²/sec (PAO4 and PAO5). Among the 1-olefins, preference is given to those which have a low degree of branching.

In particular, at least 90% by weight of the 1-olefins used for the oligomerization comprise structural units of the formula (CH₂)ₙ, where n is an integer which has a value of at least 5, for example from 5 to 20 and in particular from 6 to 12.

These include linear, i.e. unbranched, 1-olefins of the formula I
H₂C═CH—(CH₂)_{q}H I

where q+2 corresponds to the total number of carbon atoms in the olefin, 2-methyl-1-olefins of the formula II
H₂C═C(CH₃)(CH₂)ₚH II

where p+3 corresponds to the total number of carbon atoms in the olefin. Olefins of the formula II are also referred to as 2-methyl-α-olefins.

Examples of olefins of the formula I are 1-octene, 1-nonene, 1-decene, 1-dodecene, 1-tetradecene, 1-hexadecene, 1-octadecene and 1-eicosene. Examples of olefins of the formula II are 2-methyl-1-octene, 2-methyl-1-decene, 2-methyl-1-dodecene and the like.

Suitable 1-olefins also include, in principle, the dimerization products of linear 1-olefins having from 4 to 14 carbon atoms which consist in large measure, generally to an extent of >95%, of vinylidene olefins of the formula III

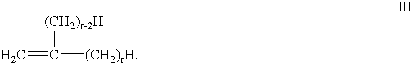

In the formula III, r corresponds to the number of carbon atoms in the linear α-olefins used for the dimerization.

If PAO4 and PAO5 are to be produced, olefins having 8 to 14, in particular from 10 to 12, carbon atoms are used for the oligomerization.

In a specific embodiment of the invention, which is of particular interest when PAO4 is to be produced, the 1-olefin to be oligomerized comprises at least 90% by weight, in particular at least 99% by weight, of 1-decene. In a further specific embodiment, which is of particular interest for the production of PAO5, the 1-olefin to be oligomerized comprises at least 90% by weight, in particular at least 99% by weight, of 1-dodecene.

In the process of the present invention, the reaction mixture usually contains no further components in addition to the 1-olefin as starting material, boron trifluoride, activator compound A1, if desired compound A2 and if desired chlorinated hydrocarbon, i.e. the proportion of other components is less than 1% by weight. It goes without saying that any extraneous components present are inert components. In another embodiment of the process of the present invention, the reaction mixture can be diluted with an inert solvent. Inert solvents are ones which do not form complexes with the boron trifluoride, for example saturated hydrocarbons such as n-hexane, n-octane, n-decane, cyclo-hexane, cyclopentane and the like. The proportion of solvent, based on the total weight of the reaction mixture (starting materials, BF₃, compound A1, A2, chlorinated hydrocarbon and solvent), is then generally from 5 to 50% by weight, in particular from 5 to 20% by weight.

In carrying out the oligomerization, the 1-olefins to be oligomerized are brought into contact with boron trifluoride and at least one protic activator and also at least one further compound selected from among aprotic organic compounds A2 and chlorinated hydrocarbons in the desired ratio. The components can be brought into contact with one another in a manner known per se; the particular measures usually depend on whether the reaction is carried out continuously or batchwise.

In a batchwise reaction, the procedure can be, for example, firstly to place the olefin to be oligomerized or part thereof, e.g. from 10 to 50% by weight, together with the compound A1 and, if desired, the compound A2 and/or the chlorinated hydrocarbon in the reaction vessel and subsequently to introduce the desired amount of boron trifluoride at a temperature below the reaction temperature, generally with good mixing being ensured. The mixture is then heated to the desired reaction temperature and this temperature is maintained until the desired conversion has been achieved. Further 1-olefin can be introduced before or during heating or during the course of the reaction. Boron trifluoride can be added as gaseous boron trifluoride or in the form of a complex of boron trifluoride and the compound A1 and, if present, the compound A2, if desired as a solution in the chlorinated hydrocarbon. Of course, it is also possible to add part of the boron trifluoride as complex and another part in gaseous form. In another variant, part of the boron trifluoride and the compound A1, if desired A2 and/or the chlorinated hydrocarbon are placed in the reactor, the 1-olefin to be oligomerized is then introduced at a temperature below the oligomerization temperature, the mixture is heated to the oligomerization temperature and further boron trifluoride is then added until the desired molar ratio of boron trifluoride to the total amount of compound A1 and A2 is in the desired range. The initial charging of the boron trifluoride can be carried out by producing a complex of boron trifluoride, the compound A1 and, if applicable, A2 in a separate reaction vessel and then placing this in the reaction vessel. Of course, it is also possible for the compound A1, if desired the compound A2 and if desired the chlorinated hydrocarbon to be initially charged and boron trifluoride then to be introduced into the reaction vessel in the desired amount. It goes without saying that these steps are carried out with mixing.

In a continuous reaction, the 1-olefin to be oligomerized, boron trifluoride, the compound A1 and the compound A2 and/or the chlorinated hydrocarbon are generally fed continuously in the desired ratios into a reaction zone and oligomerization product is taken off continuously. The boron trifluoride can be introduced wholly or partly as a mixture with the compound A1 and/or the compound A2. BF₃ and the compound A1 and, if desired, the compound A2 are frequently introduced into the reactor separately, but preferably in close proximity to one another, ideally via multifluid nozzles.

The reaction temperature required for the oligomerization is usually in the range from −20° C. to +60° C. and preferably in the range from +0 to +40° C. Higher temperatures can be employed in principle, but are generally not necessary and are disadvantageous if a high viscosity index is to be achieved. The reaction is usually carried out at atmospheric pressure or at slightly superatmospheric pressure, e.g. up to 1.5 bar, with a BF₃ partial pressure of 1.3 bar, preferably 1.2 bar, in particular 1.1 bar and especially atmospheric pressure (1013 mbar), normally not being exceeded. Higher pressures, in particular higher BF₃ pressures of >1.5 bar or even >2 bar, as required in the prior art, are not necessary for achieving satisfactory conversions and are disadvantageous in terms of the viscosity index of the products obtained in the oligomerization.

When the desired degree of oligomerization has been achieved, the reaction is stopped in a manner known per se by deactivation of the boron trifluoride. The deactivation is usually carried out by addition of sufficient amounts of a termination agent. This term refers to compounds which form complexes with boron trifluoride. The amount of termination agent is chosen so that the molar ratio of all compounds which form complexes with boron trifluoride exceeds 1.25:1, in particular 2:1 and particularly preferably 3:1. Suitable termination agents are, in particular, water, methanol, ethanol, acetonitrile, sodium fluoride, aqueous ammonia and sodium hydroxide. Not least for cost reasons, water is preferably used as termination agent for the deactivation.

However, if the boron trifluoride is to be recirculated to the reaction, it is advisable to bring about the termination of the reaction by addition of sufficient amounts of compound A1. For example, in the case of methanol, a BF₃-rich phase separates out and can, if desired after addition of further BF₃, be returned to the oligomerization reaction. It goes without saying that only just that amount of compound A1 which is sufficient to produce separation of a BF₃-rich phase is added. In particular, in the case of a continuous reaction, just that amount of BF₃-rich phase which contains an amount of compound A1 corresponding to the amount of compound A1 required for the oligomerization is separated out.

If phase separation into a complex-rich phase and a low-BF₃ phase occurs in the reaction, it is advisable to separate off and recirculate the BF₃-rich phase, which contains comparatively little reaction product, first. The phase depleted in the complex is subsequently treated with the desired amount of termination agent. The BF₃-rich phase is separated off in a manner known per se by phase separation in phase separation vessels, with or without aids such as coalescing filters or coalescence tubes.

After the reaction has been stopped, a customary work-up is carried out. For this purpose, the reaction mixture can, for example, be washed with water to remove inorganic compounds and any compounds A1. It is subsequently worked up by distillation to separate off residual amounts of solvent, unreacted 1-olefins and 1-olefin dimers. The residue then comprises the desired olefin oligomers. If the viscosity of the residue is too low, the viscosity can be increased by distilling off further trimer. If it is too high, some trimer distillate can be added. In any case, the major part of the product, i.e. more than 80% and in particular more than 90%, is a bottom product and not a distillate.

The oligomerization product obtained generally still has ethylenically unsaturated double bonds which have adverse effects when it is used as base oil for motor oils. The oligomerization product obtained is therefore generally subjected to a hydrogenation to saturate these double bonds. The hydrogenation is usually carried out as a catalytic hydrogenation over a transition metal catalyst using methods known per se, as are also cited in the prior art discussed above. Suitable hydrogenation catalysts generally comprise at least one transition metal of groups VI, VIII and I, e.g. platinum, palladium, nickel, copper, chromium or combinations of these metals, as catalytically active species. The catalytically active species is preferably used as a heterogeneous catalyst, in particular in supported form. Possible support materials are, for example, activated carbon, kieselguhr, aluminum oxide, zeolites, TiO₂, ZrO₂ and the like. Examples of suitable catalysts are platinum on activated carbon, palladium on activated carbon, Raney nickel, nickel on silica gel, copper chromite, copper/palladium catalysts supported on aluminum oxide, and the like. The hydrogen partial pressure required for the hydrogenation is generally in the range from 1 to 600 bar, preferably from 5 to 200 bar. The hydrogenation temperature is frequently in the range from 30 to 300° C. and in particular in the range from 100 to 200° C. The hydrogenation is generally carried out until more or less complete saturation of the olefinic double bonds has been achieved. The proportion of unhydrogenated double bonds can be determined in a simple manner via the iodine number or the bromine number (using a method based on DIN 51774). The hydrogenation products usually have iodine numbers of less than 1.0, in particular less than 0.2.

The measures provided according to the present invention of adding an aprotic organic compound A2 and/or a chlorinated hydrocarbon to the reaction mixture enable the consumption of boron trifluoride in the oligomerization to be reduced significantly compared to the processes of the prior art. The BF₃ consumption is generally less than 2% by weight and in particular less than 1.5% by weight, based on the 1-olefins to be oligomerized. The conversions of 1-olefins achieved are significantly above the conversions achieved without addition of compound A2 and/or a chlorinated hydrocarbon. Surprisingly, the process of the present invention also makes it possible to prepare low-viscosity products having viscosities of from about 4 to 5 mm²/sec, i.e. PAO4 and PAO5, in a targeted manner, without formation of coproducts having higher viscosities. This is particularly surprising since a specific preparation of PAO5 has not been described hitherto. Isolation of these components from the oligomerization mixture by distillation is not necessary, unlike the case of the prior art. In addition, the viscosity indices of the oligomers obtained according to the present invention are, surprisingly, significantly greater than the viscosity indices of products which are obtained from oligomerization products by distillation. The oligomers obtainable by the process of the present invention are therefore novel and likewise subject matter of the invention. They generally have viscosities (determined in accordance with DIN 51562-1 to 4 by means of an Ubbelohde viscometer at 100° C.) in the range from 3.5 mm²/sec to 8 mm²/sec at a viscosity index (determined in accordance with ISO 2909 from the viscosities at 40° C. and 100° C.) of generally from ≧128 to 140 (at a viscosity of about 4 mm²/sec) and from ≧135 to 150 (at a viscosity of from 5 mm²/sec to 8 mm²/sec).

The following examples illustrate the invention.

I. Analysis

Viscosimetric Examination:

The viscosities of the oligomerization products were determined in accordance with DIN 51562-1 to 4 by means of an Ubbelohde viscometer at the temperature indicated in each case. The viscosity reported is the kinematic viscosity. The viscosity index was determined in a customary manner from the viscosity values at 40° C. and 100° C. (in accordance with ISO 2909).

The abbreviations below are used in the following:

TABLE 1
uz,13/20 Donor numbers

| Abbreviation | Chemical name | Donor number* |
|---|---|---|
| BuOH | n-Butanol | 29 |
| PeOH | n-Pentanol | 25 |
| MeOH | Methanol | 30 |
| EtOH | Ethanol | 32 |
| i-PrOH | Isopropanol | 36 |
| n-PrOH | n-Propanol | 33** |
| IBTBE | Isobutyl tert-butyl ether | 16** |
| MTBE | Methyl tert-butyl ether | 14** |
| DHE | Dihexyl ether | 19** |
| DOE | Dioctyl ether | 19** |
| DIPE | Diisopropyl ether | 19** |

*by the method of Gutmann (cf. Y. Marcus, Chemical Society Reviews, 1993, p. 410)
**own measurements using the method described by Gutmann

II. Oligomerization of 1-olefins

General Method A:

The amounts of compound A1 and, where applicable, A2 and, where applicable, dichloromethane indicated in table 2 were placed in a 11 four-neck flask fitted with thermometer, dropping funnel, gas inlet tube for boron trifluoride, gas outlet tube with bubble counter and magnetic stirrer and the mixture was cooled to −10° C. 10 mmol of boron trifluoride were passed in while maintaining the temperature. 140 g of 1-decene were then added with cooling. The cooling bath was removed and the mixture was allowed to warm to 30° C. 10 mmol (or 5 mmol—see table 2) of boron trifluoride were then passed into the reaction mixture over a period of 10 minutes while stirring vigorously. A temperature of 30° C. was subsequently maintained for 2 hours. 200 ml of ice water were then added at 30° C. while stirring vigorously. After allowing the phases to separate for 15 minutes, the aqueous phase was separated off and the organic phase was washed twice with 200 ml each time of water, dried over sodium sulfate and subsequently freed of solvent, 1-decene and decene dimers on a rotary evaporator. For this purpose, the pressure was slowly reduced to 0.1 mbar and the temperature was increased continuously to 150° C. After 30 minutes at 150° C. and 0.1 mbar, a product containing less than 2% by weight of dimer was obtained. The viscosity of this was determined in the above-described manner. The viscosity at 100° C. and the viscosity index [VI] are reported in table 3.

TABLE 2

| Ex- | Compound A1 | | Compound A2 | | | Dichloro- | |
|---|---|---|---|---|---|---|---|
| am- | | Amount | | Amount | BF₃ | methane | Yield |
| ple | Type | [mmol] | Type | [mmol] | [mmol] | [g] | [%]* |
| C1 | PeOH | 10 | — | — | 20 | — | 11 |
| 1 | PeOH | 5 | IBTBE | 5 | 20 | — | 29 |
| 2 | PeOH | 6 | DHE | 4 | 20 | — | 50 |
| 3 | PeOH | 5 | DHE | 5 | 20 | — | 87 |
| C2 | BuOH | 10 | — | — | 20 | — | 40 |
| 4 | BuOH | 5 | MTBE | 5 | 20 | — | 81 |
| 5 | MeOH | 5 | DHE | 5 | 20 | — | 42 |
| 6 | PeOH | 5 | DOE | 2.5 | 20 | 15 | 86 |
| 7 | PeOH | 5 | DIPE | 2.5 | 20 | 15 | 40 |
| 8 | BuOH | 5 | DOE | 2.5 | 20 | 15 | 86 |
| 9 | BuOH | 15 | — | — | 20 | 15 | 86 |
| 10 | BuOH | 7.5 | — | — | 15 | 15 | 72 |
| 11 | BuOH | 7.5 | DOE | 2.5 | 20 | 15 | 93 |
| 12** | n-PrOH | 15 | — | — | 20 | 15 | n.d. |
| 13** | i-PrOH | 15 | — | — | 20 | 15 | 83 |
| 14** | EtOH | 15 | — | — | 20 | 15 | 87 |

*% by weight, based on 1-decene used;
n.d. = not determined
**emulsion

TABLE 3

| | Viscosity at 100° C. | |
|---|---|---|
| Example | [mm²/sec] | VI* |
| C1 | n.d. | n.d. |
| 1 | 3.89 | 131 |
| 2 | 3.96 | 133 |
| 3 | 5.12 | 136 |
| C2 | 3.98 | 129 |
| 4 | 4.03 | 133 |
| 5 | 3.94 | 132 |
| 6 | 6.17 | 141 |
| 7 | n.d. | n.d. |
| 8 | 7.54 | 147 |
| 9 | 5.89 | 143 |
| 10 | 4.41 | 133 |
| 11 | 6.31 | 143 |

*Viscosity index (in accordance with ISO 2909)

General Method B

In a 11 four-neck flask fitted with thermometer, dropping funnel, gas inlet tube, gas outlet tube with bubble counter and magnetic stirrer, 210 g of 1-decene were admixed with the amount indicated in table 4 of a boron trifluoride-methanol complex (1:1 mol/mol). The remaining amount of boron trifluoride was then passed in over a period of 10 minutes. While stirring vigorously, the reaction mixture was maintained at 30° C. for 2 hours. The stirrer was then switched off and the phases were allowed to separate for 30 minutes. The lower, complex-rich phase was taken off and the upper phase (product phase) was washed with water and worked up in the manner described for method A. The amounts used and results are reported in tables 4 and 5.

TABLE 4

| | | | | | | BF₃ | |
|---|---|---|---|---|---|---|---|
| | Compound A1 | | Compound A2 | | | loss | |
| Ex- | | Amount | | Amount | BF₃ | [% by | Yield |
| ample | Type | [mmol] | Type | [mmol] | [mmol] | weight] | [%] |
| 16 | MeOH | 10 | MTBE | 100 | 200 | 0.8 | 94 |
| 17 | MeOH | 4 | MTBE | 40 | 90 | 0.8 | 91 |

TABLE 5

| | Viscosity at 100° C. | |
|---|---|---|
| Example | [mm²/sec] | VI* |
| 16 | 4.33 | 129 |
| 17 | 3.92 | 126 |

*Viscosity index (in accordance with ISO 2909)

We claim:

1. A process for preparing synthetic hydrocarbons, which comprises the oligomerization of 1-olefins having from 8 to 30 carbon atoms in the presence of boron trifluoride and at least one protic activator compound A1, wherein the reaction is carried out in the presence of at least one further compound selected from among:
aprotic organic compounds A2 which are selected from among ethers, comprising at least one ether oxygen atom, and
halogenated hydrocarbons having from 1 to 6 carbon atoms and 1, 2 or 3 halogen atoms,
where the molar ratio of boron trifluoride to the total amount of compounds A1 and A2 is in the range from 0.8:1 to 4:1, wherein the absolute pressure of boron trifluoride in the reactor does not exceed a value of 1.3 bar.

2. A process as claimed in claim 1, wherein the oligomerization is carried out at a boron trifluoride concentration below the saturation concentration of boron trifluoride in the reaction mixture at a BF₃ pressure of 1 bar and the reaction temperature.

3. A process as claimed in either of the preceding claims, wherein the concentration of boron trifluoride in the reaction mixture is in the range from 0.5 to 2% by weight.

4. A process as claimed in any of the preceding claims, wherein the activator compound A1 is selected from among alkanols having from 1 to 10 carbon atoms.

5. A process as claimed in any of the preceding claims, wherein the compound A2 is selected from among dialkyl ethers having at least 5 carbon atoms.

6. A process as claimed in any of the preceding claims, wherein the oligomerization is carried out in the presence of from 2 to 20% by weight, based on the total weight of the reaction mixture, of at least one chlorinated hydrocarbon.

7. A process as claimed in any of the preceding claims, wherein the oligomerization is carried out in the presence of a compound A2 and the molar ratio of compound A1 to compound A2 is in the range from 5:1 to 1:20.

8. A process as claimed in claim 1, wherein the mean number of carbon atoms in the compounds A1 and A2, weighted according to the mole fractions of A1 and A2, is in the range from 4 to 7.

9. A process as claimed in claim 8, wherein the activator compound A1 is selected from among alkanols having from 2 to 6 carbon atoms.

10. A process as claimed in any of claims 1 to 7, wherein the type and amount of compound A1, if desired A2 and if desired the type and amount of the chlorinated hydrocarbon are selected so that phase separation into a first phase having a high BF₃ concentration and a second phase having a low BF₃ concentration occurs, with the solubility of BF₃ in the second phase being less than 1% by weight, based on the weight of the second phase.

11. A process as claimed in claim 10, wherein the activator compound A1 is selected from among water, methanol and hydrogen fluoride and the further compound or compounds is/are selected from among dialkyl ethers having from 5,to 10 carbon atoms as compound A2.

12. A process as claimed in claim 11, wherein the molar ratio of compound A1 to compound A2 is in the range from 1:2 to 1:20.

13. A process as claimed in claim 10, wherein the oligomerization is carried out in the absence of a compound A2 and the compound A1 is selected from among ethanol, isopropanol and n-propanol.

14. A process as claimed in any of the preceding claims, wherein at least 90% by weight of the 1-olefins used for the oligomerization comprises structural units of the formula (CH₂)ₙ, where n is an integer of at least 5.

15. A process as claimed in claim 14, wherein at least 90% of the 1-olefins used for the oligomerization is selected from among unbranched α-olefins and 2-methyl-α-olefins.

16. A process as claimed in any of the preceding claims, wherein the 1-olefins to be oligomerized have from 8 to 12 carbon atoms.

17. A process as claimed in any of the preceding claims, wherein the 1-olefins to be oligomerized comprise at least 90% by weight of 1-decene.

18. A process as claimed in any of the preceding claims, wherein the 1-olefins to be oligomerized comprise at least 90% by weight of 1-dodecene.

19. A process as claimed in any of the preceding claims, wherein the oligomerization is carried out at from −20 to +40° C.

20. A process as claimed in any of the preceding claims, wherein the oligomerization product is subjected to a catalytic hydrogenation.

21. A synthetic hydrocarbon obtainable by a process as claimed in any of claims 1 to 20.
